(19) United States
(12) Patent Application Publication
(10) Pub. No.: US 2024/0319202 A1
(43) Pub. Date: Sep. 26, 2024

(54) METHOD FOR EVALUATING AGGREGATION STATE OF AGGREGATIVE PROTEIN

(71) Applicant: HAMAMATSU PHOTONICS K.K., Hamamatsu-shi (JP)

(72) Inventors: Gen TAKEBE, Hamamatsu-shi (JP); Shigetoshi OKAZAKI, Hamamatsu-shi (JP)

(73) Assignee: HAMAMATSU PHOTONICS K.K., Hamamatsu-shi (JP)

(21) Appl. No.: 18/591,189

(22) Filed: Feb. 29, 2024

(30) Foreign Application Priority Data
Mar. 22, 2023 (JP) 2023-045508

(51) Int. Cl. G01N 33/68; C09B 11/06; C09K 11/06; G01N 21/31; G01N 21/64

(52) U.S. Cl. CPC G01N 33/68; C09B 11/06; C09K 11/06; G01N 21/3103; G01N 21/6428; C09K 2211/1007; C09K 2211/1018; G01N 2021/6439

(57) ABSTRACT

A method for evaluating an aggregation state of aggregative proteins in a solution is disclosed, the method comprising evaluating an aggregation state of aggregative proteins using, as an index, light-absorption/fluorescence characteristic(s) of a certain xanthene dye in a solution comprising the aggregative proteins and the xanthene dye or a salt thereof.

### TECHNICAL FIELD

The present disclosure relates to a method for evaluating an aggregation state of aggregative proteins.

### BACKGROUND

Certain proteins are known to aggregate with each other in an aqueous solvent to form aggregates. As the aggregation state of proteins, there are a plurality of states such as dimers, oligomers, protofibrils, and fibrils as aggregates, in addition to a non-aggregating monomer state. In addition, amyloid fibrils, amorphous aggregates, and the like are known as forms of formed aggregates.

Aggregates of proteins are known to be associated with diseases, and extensive research is being conducted with the aim of using them as diagnostic markers or therapeutic targets for diseases. Examples of typical proteins known to be associated with diseases include amyloid β and tau protein. In the brain nervous tissue of patients with Alzheimer's disease, fibrils of amyloid β, and aggregates of tau protein that is abnormally phosphorylated as the amount of intracellular amyloid β increases are observed. In particular, it is thought that aggregates of tau protein induce nerve cell damage and cell death, thereby causing a decline in cognitive ability. The following hypothesis is called the “amyloid β hypothesis:” when the formation of aggregates of amyloid β in brain nervous tissue can be prevented, this can also inhibit an increase of intracellular amyloid β, which can inhibit a decline in cognitive ability. Based on the amyloid R hypothesis, development of therapeutic agents for Alzheimer's disease which target the aggregates of amyloid β is underway.

When elucidating the environmental dependence or function of the aggregation state of proteins, an approach of evaluating the aggregation state of proteins in a solution is useful. Regarding the evaluation of the aggregation state of proteins in a solution, for example, Patent Document 1 (Japanese Unexamined Patent Publication No. 2019-527353) discloses a method for detecting the presence of alpha-synuclein aggregates in a biological specimen, the method including mixing a biological specimen with a reaction specimen containing a bead group, thioflavin T as a fluorescent dye, and alpha-synuclein or a fragment or mutant thereof. In the method, a significant increase in the fluorescence of the reaction mixture during incubation indicates the presence of alpha-synuclein aggregates in the biological specimen.

### SUMMARY

Although the aggregation state of proteins in a solution has traditionally been evaluated using the turbidity of the solution as an index, because its dynamic range is small, only a part of time-dependent changes in the aggregation state of proteins in the solution may be identified.

An object of the present disclosure is to provide a method for evaluating an aggregation state of aggregative proteins in a solution.

The inventors of the present invention completed the present disclosure by finding that an aggregation state of aggregative proteins in a solution can be evaluated using, as an index, light-absorption/fluorescence characteristic(s) of a xanthene dye in a solution containing the aggregative proteins and the xanthene dye.

The present disclosure relates to the following [1] to [17].

[1] A method for evaluating an aggregation state of aggregative proteins in a solution, the method comprising evaluating an aggregation state of aggregative proteins using, as an index, light-absorption/fluorescence characteristic(s) of a xanthene dye in a solution comprising the aggregative proteins and the xanthene dye or a salt thereof.

[2] A method for evaluating an aggregation state of aggregative proteins in a solution, the method comprising evaluating an aggregation state of aggregative proteins using, as an index, light-absorption/fluorescence characteristic(s) of a xanthene dye in a solution containing the aggregative proteins and containing the xanthene dye or a salt thereof,
wherein the xanthene dye is represented by Formula (I):

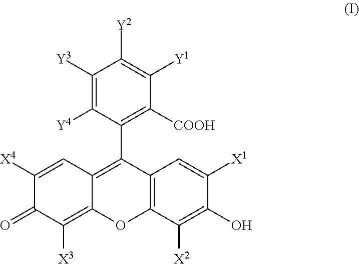

wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

[3] The method according to [1] or [2], in which the aggregation state is evaluated based on a relationship between a time from when the aggregative proteins are brought into an aggregatable state in the solution, and the light-absorption/fluorescence characteristic(s) of the xanthene dye.

[4] The method according to any one of [1] to [3], wherein X¹ to X⁴ are each independently —H, —OH, —NO₂, —F, —Cl, —Br, or —I, provided that at least two of X¹ to X⁴ are —Br or —I; and Y¹ to Y⁴ are each independently —H or —Cl.

[5] The method according to any one of [1] to [3], wherein X¹ to X⁴ are each independently —Br or —I; and Y¹ to Y⁴ are each independently —H or —Cl.

[6] The method according to any one of [1] to [3], wherein the xanthene dye or a salt thereof is rose bengal, eosin Y, or erythrosin B.

[7] The method according to any one of [1] to [6], wherein the light-absorption/fluorescence characteristic(s) includes at least one selected from the group consisting of a fluorescence spectrum, a fluorescence intensity, a maximum fluorescence wavelength, an absorption spectrum, an absorbance, and a maximum absorption wavelength.

[8] The method according to any one of [1] to [6], wherein the light-absorption/fluorescence characteristic(s) includes at least one selected from the group consisting of a fluorescence spectrum, a fluorescence intensity, and a maximum fluorescence wavelength.

[9] The method according to any one of [1] to [6], wherein the light-absorption/fluorescence characteristic(s) includes at least one selected from the group consisting of a fluorescence spectrum, a maximum fluorescence intensity, and a maximum absorbance.

[10] The method according to any one of [1] to [6], wherein the light-absorption/fluorescence characteristic(s) is a fluorescence spectrum or a fluorescence intensity as, and wherein the aggregation state of the aggregative proteins is evaluated based on a relationship in which a maximum fluorescence intensity or a fluorescence intensity at a predetermined fluorescence wavelength decreases and thereafter increases with respect to a time from when the aggregative proteins are brought into an aggregatable state in the solution, the predetermined fluorescence wavelength being a wavelength which can be determined depending on the xanthene dye and the aggregative proteins.

[11] A kit for use in the method according to any one of [1] to [10], the kit comprising: the xanthene dye or the salt thereof; and an instruction manual describing evaluation of an aggregation state of aggregative proteins in a solution using light-absorption/fluorescence characteristic(s) of the xanthene dye as a criterion,
wherein the xanthene is represented by Formula (I):

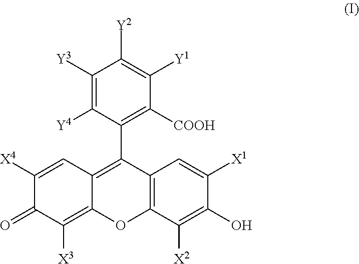

wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

[12] A kit for use in the method according to any one of [1] to [10], the kit comprising: the xanthene dye or the salt thereof; and at least one selected from the group consisting of an antioxidant substance, a pH adjuster, and a buffering agent,
wherein the xanthene is represented by Formula (I):

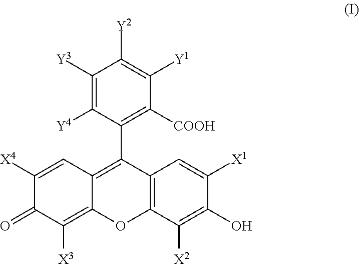

wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.)

[13] A method for screening drugs that induce a change in an aggregation state of aggregative proteins or induce decomposition of an aggregate of the aggregative proteins, the method comprising evaluating the aggregation state of the aggregative proteins in a solution according to the method according to any one of [1] to [10].

[14] A method for evaluating drugs that induce a change in an aggregation state of aggregative proteins or induce decomposition of an aggregate of the aggregative proteins, the method comprising evaluating the aggregation state of the aggregative proteins in a solution according to the method according to any one of [1] to [10].

[15] A screening kit for use in the method according to [13], the screening kit containing: the xanthene dye or the salt thereof; and the aggregative protein,
wherein the xanthene is represented by Formula (I):

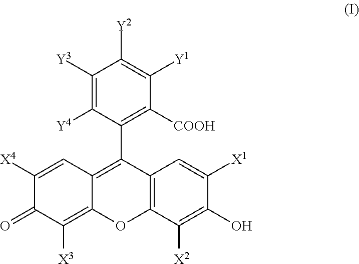

wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

[16] An evaluation kit for use in the method according to [14], the evaluation kit containing: the xanthene dye or the salt thereof; and the aggregative protein,
wherein the xanthene is represented by Formula (I):

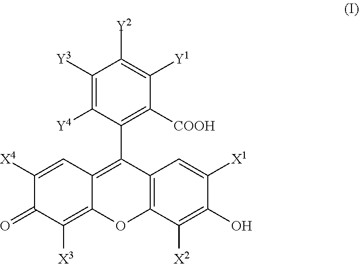

wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

[17] A method for isolating an aggregate of aggregative proteins, the method including isolating an aggregate of aggregative proteins from a solution using, as an index, the aggregation state of the aggregative proteins in the solution evaluated according to the method according to any one of [1] to [10].

According to one embodiment of the present disclosure, an aggregation state of aggregative proteins in a solution can be evaluated using light-absorption/fluorescence characteristic(s) of a xanthene dye as an index.

According to another embodiment of the present disclosure, the aggregation state of the aggregative proteins can be evaluated based on a relationship in which an absorbance and/or a fluorescence intensity decreases and thereafter increases with respect to a time from when the aggregative proteins are brought into an aggregatable state in the solution.

### BRIEF DESCRIPTION OF THE DRAWINGS

FIG. 1 is a diagram showing absorption spectra at various heating-stirring times (heat denaturation times) when the aggregation state of lysozyme was evaluated using the light-absorption/fluorescence characteristic of rose bengal as an index in Example 1.

FIG. 2 is a diagram showing the results of plotting with a horizontal axis representing a heat denaturation time and a vertical axis representing a maximum absorption wavelength or a maximum absorbance when the aggregation state of lysozyme was evaluated using the light-absorption/fluorescence characteristic of rose bengal as an index in Example 1.

FIG. 3 is a diagram showing fluorescence spectra at various heating-stirring times (heat denaturation times) when the aggregation state of lysozyme was evaluated using the light-absorption/fluorescence characteristic of rose bengal as an index in Example 1.

FIG. 4 is a diagram showing the results of plotting with a horizontal axis representing a heat denaturation time and a vertical axis representing a maximum fluorescence wavelength or a maximum fluorescence intensity when the aggregation state of lysozyme was evaluated using the light-absorption/fluorescence characteristic of rose bengal as an index in Example 1.

FIG. 5 is a diagram showing absorption spectra at various heating-stirring times (heat denaturation times) when the aggregation state of insulin was evaluated using the light-absorption/fluorescence characteristic of rose bengal as an index in Example 2.

FIG. 6 is a diagram showing the results of plotting with a horizontal axis representing a heat denaturation time and a vertical axis representing a maximum absorption wavelength or a maximum absorbance when the aggregation state of insulin was evaluated using the light-absorption/fluorescence characteristic of rose bengal as an index in Example 2.

FIG. 7 is a diagram showing fluorescence spectra at various heating-stirring times (heat denaturation times) when the aggregation state of insulin was evaluated using the light-absorption/fluorescence characteristic of rose bengal as an index in Example 2.

FIG. 8 is a diagram showing the results of plotting with a horizontal axis representing a heat denaturation time and a vertical axis representing a maximum fluorescence wavelength or a maximum fluorescence intensity when the aggregation state of insulin was evaluated using the light-absorption/fluorescence characteristic of rose bengal as an index in Example 2.

FIG. 9 is a diagram showing absorption spectra at various heating-stirring times (heat denaturation times) when the aggregation state of horseradish peroxidase was evaluated using the light-absorption/fluorescence characteristic of rose bengal as an index in Example 3.

FIG. 10 is a diagram showing the results of plotting with a horizontal axis representing a heat denaturation time and a vertical axis representing a maximum absorption wavelength or a maximum absorbance when the aggregation state of horseradish peroxidase was evaluated using the light-absorption/fluorescence characteristic of rose bengal as an index in Example 3.

FIG. 11 is a diagram showing fluorescence spectra at various heating-stirring times (heat denaturation times) when the aggregation state of horseradish peroxidase was evaluated using the light-absorption/fluorescence characteristic of rose bengal as an index in Example 3.

FIG. 12 is a diagram showing the results of plotting with a horizontal axis representing a heat denaturation time and a vertical axis representing a maximum fluorescence wavelength or a maximum fluorescence intensity when the aggregation state of horseradish peroxidase was evaluated using the light-absorption/fluorescence characteristic of rose bengal as an index in Example 3.

FIG. 13 is a diagram showing absorption spectra at various heating-stirring times (heat denaturation times) when the aggregation state of lysozyme was evaluated using the light-absorption/fluorescence characteristic of eosin Y as an index in Example 4.

FIG. 14 is a diagram showing the results of plotting with a horizontal axis representing a heat denaturation time and a vertical axis representing a maximum absorption wavelength or a maximum absorbance when the aggregation state of lysozyme was evaluated using the light-absorption/fluorescence characteristic of eosin Y as an index in Example 4.

FIG. 15 is a diagram showing fluorescence spectra at various heating-stirring times (heat denaturation times) when the aggregation state of lysozyme was evaluated using the light-absorption/fluorescence characteristic of eosin Y as an index in Example 4.

FIG. 16 is a diagram showing the results of plotting with a horizontal axis representing a heat denaturation time and a vertical axis representing a maximum fluorescence wavelength or a maximum fluorescence intensity when the aggregation state of lysozyme was evaluated using the light-absorption/fluorescence characteristic of eosin Y as an index in Example 4.

FIG. 17 is a diagram showing absorption spectra at various heating-stirring times (heat denaturation times) when the aggregation state of lysozyme was evaluated using the light-absorption/fluorescence characteristic of erythrosin B as an index in Example 5.

FIG. 18 is a diagram showing the results of plotting with a horizontal axis representing a heat denaturation time and a vertical axis representing a maximum absorption wavelength or a maximum absorbance when the aggregation state of lysozyme was evaluated using the light-absorption/fluorescence characteristic of erythrosin B as an index in Example 5.

FIG. 19 is a diagram showing fluorescence spectra at various heating-stirring times (heat denaturation times) when the aggregation state of lysozyme was evaluated using the light-absorption/fluorescence characteristic of erythrosin B as an index in Example 5.

FIG. 20 is a diagram showing the results of plotting with a horizontal axis representing a heat denaturation time and a vertical axis representing a maximum fluorescence wavelength or a maximum fluorescence intensity when the aggregation state of lysozyme was evaluated using the light-absorption/fluorescence characteristic of erythrosin B as an index in Example 5.

### DETAILED DESCRIPTION

Although embodiments of the present disclosure will be described below, the present disclosure is not limited to the following embodiments.

A first aspect of the present disclosure is a method for evaluating an aggregation state of aggregative proteins in a solution, the method including evaluating an aggregation state of aggregative proteins using, as an index, light-absorption/fluorescence characteristic(s) of a xanthene dye in a solution containing the aggregative proteins and containing the xanthene dye or a salt thereof.

According to the first aspect of the present disclosure, the solution, for which the light-absorption/fluorescence characteristic(s) as an index of evaluation is measured, is an aqueous solution containing at least the aggregative proteins and the xanthene dye. In other words, a solvent in the solution, for which the light-absorption/fluorescence characteristic(s) as an index of evaluation is measured, is an aqueous solvent. Examples of the aqueous solvents include water, and a mixed solvent of water and an organic solvent. The organic solvent may be any organic solvent as long as it is miscible with water, and examples thereof include dimethyl sulfoxide, ethanol, methanol, acetone, N,N-dimethylformamide, and 1,4-dioxane, among which dimethyl sulfoxide or ethanol is preferable. Furthermore, the content of water in the aqueous solvent may be 50 v/v % or more, 70 v/v % or more, 90 v/v % or more, 95 v/v % or more, 98 v/v % or more, 99 v/v % or more, or 100 v/v %, and is preferably 90 v/v % or more, and more preferably 95 v/v % or more, for example.

In the present disclosure, the aggregative proteins mean proteins that aggregate with each other in an aqueous solvent to form an aggregate. As stages of the aggregation state, examples of aggregates include dimers, oligomers, protofibrils, and fibrils, in addition to monomers not forming aggregates. The aggregative proteins gradually aggregate into dimers, oligomers, and the like from the monomer state before aggregation to finally form fibrils. Furthermore, examples of forms of formed aggregates include amyloid fibrils and amorphous aggregates. The proteins according to one embodiment of the present disclosure may be proteins including a glycoprotein, or may be proteins excluding a glycoprotein.

As the aggregative proteins, there are proteins that aggregate with each other under physiological conditions, and there are also proteins that aggregate under specific conditions in which denaturation, a change in chemical structure, or the like occurs, among which any of them may be used as the aggregative proteins according to the present disclosure.

Examples of proteins that aggregate with each other under physiological conditions include amyloid β protein (Aβ). Amyloid β protein is a protein produced when amyloid precursor protein (APP) is cleaved by β-secretase and γ-secretase. Fibrils of amyloid β protein are known to be present in large amounts in the brain of patients with Alzheimer's disease and are being studied as one of the therapeutic targets for cognitive disorders. For example, lecanemab, which was approved by the United States Food and Drug Administration (FDA) on Jan. 6, 2023 as a therapeutic agent for Alzheimer's disease, is an anti-amyloid β protofibril antibody.

Examples of proteins that aggregate when a change in chemical structure occurs include tau protein. Tau protein is a microtubule-binding protein mainly present in nerve cells. Tau protein is present by being bound to microtubules under physiological conditions, but when excessively phosphorylated, its binding properties to microtubules decrease, and its aggregating properties increase. It is known that in the brain of patients with Alzheimer's disease, hyperphosphorylated tau protein forms aggregates (which is a phenomenon also called “neurofibrillary tangle”). These aggregates of tau protein are being studied as one of potential therapeutic targets for cognitive disorders.

Examples of proteins that aggregate when denaturation occurs include lysozyme. Lysozyme is a type of hydrolytic enzymes contained in human nasal discharge, chicken egg whites, and the like. It is known that, when lysozyme is denatured by heating or the like under acidic conditions, its aggregating properties increase, and amyloid is formed as the aggregate thereof. Therefore, denatured lysozyme is being widely used as a model for analyzing the formation mechanism of the aggregates of amyloid β protein, evaluating cytotoxicity thereof, and the like.

In one embodiment of the present disclosure, the concentration of the aggregative proteins in the solution, for which the light-absorption/fluorescence characteristic(s) as an index of evaluation is measured, may be 0.1 to 100 mg/mL, 1.0 to 75 mg/mL, 5.0 to 50 mg/mL, or 10 to 35 mg/mL, for example.

In the present disclosure, the xanthene dye means a compound represented by any of the following structural formulas.

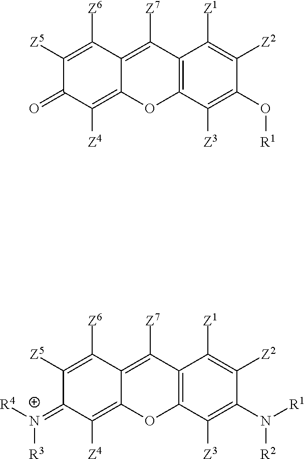

(In the formulas, Z¹ to Z⁷ each independently represent hydrogen or a monovalent substituent, and R¹ to R⁴ each independently represent hydrogen or an alkyl group.)

Examples of the xanthene dye in the present disclosure include fluorescein, rhodamine B, rhodamine 6G, rhodamine 19, phenylfluorone, rose bengal (Acid Red 94), eosin Y (tetrabromofluorescein, Solvent Red 43), Acid Red 87 (eosin Y disodium salt), erythrosin B (Acid Red 51), phloxine B (Acid Red 92), tetrabromofluorescein potassium, 2′,4′,5′,7′-tetrabromo-3,4,5,6-tetrachlorofluorescein (Solvent Red 48), tetraiodofluorescein (iodeosin), eosin B (Acid Red 91), dibromofluorescein (Solvent Red 72), and diiodofluorescein (Solvent Red 73).

In one embodiment of the present disclosure, the xanthene dye may be represented by Formula (I) below:

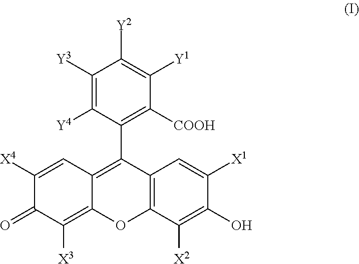

In one embodiment, in Formula (I), X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl. In a preferable embodiment, X¹ to X⁴ are each independently —H, —OH, —NO₂, —F, —Cl, —Br, or —I, provided that at least two or three of X¹ to X⁴ may be —Br or —I. In a more preferable embodiment, X¹ to X⁴ may each independently be —Br or —I. In addition, Y¹ to Y⁴ may each independently be —H or —Cl. In a particular embodiment, in Formula (I), X¹ to X⁴ may each independently be —Br or —I; and Y¹ to Y⁴ may each independently be —H or —Cl. In these cases, X¹ to X⁴ and Y¹ to Y⁴ in the formula may or may not be the same.

The xanthene dye according to one embodiment of the present disclosure is present in various equilibrium states in an aqueous solvent. In other words, the xanthene dye represented by Formula (I) above may be expressed as structures shown below, for example, as one state in the solution, but all of these represent the xanthene dye represented by Formula (I) above.

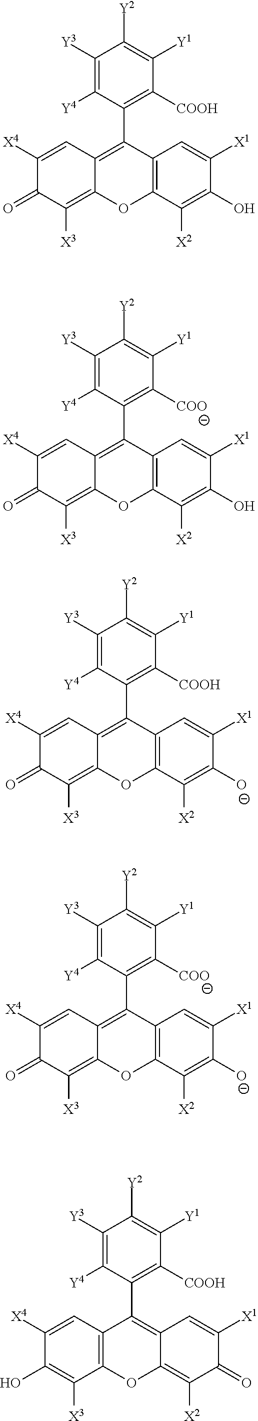

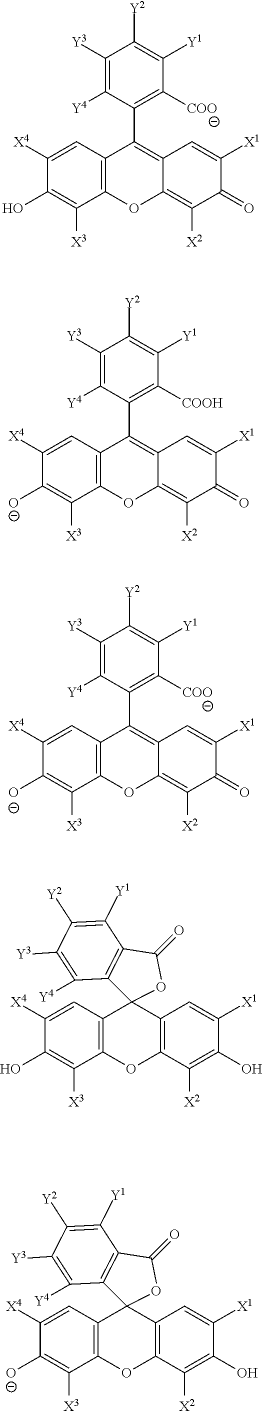

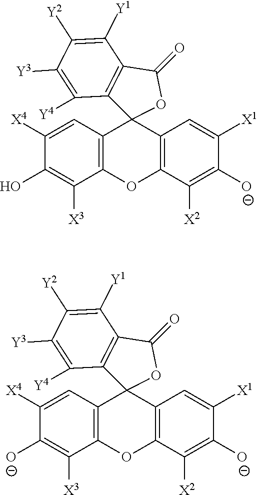

The solution according to one embodiment of the present disclosure, for which the light-absorption/fluorescence characteristic(s) as an index of evaluation is measured, may contain a free form of the xanthene dye, or may contain a salt of the xanthene dye. Counter cations and counter anions in the salt are not limited, and examples thereof include lithium ions, sodium ions, potassium ions, ammonium ions, fluoride ions, chloride ions, bromide ions, iodide ions, and hydroxide ions. When the xanthene dye according to one embodiment of the present disclosure is represented by Formula (I) above, examples of preferable counter cations include sodium ions. For example, a sodium salt of the xanthene dye represented by Formula (I) above is represented by one of the following structural formulas, all of which represent the same compound.

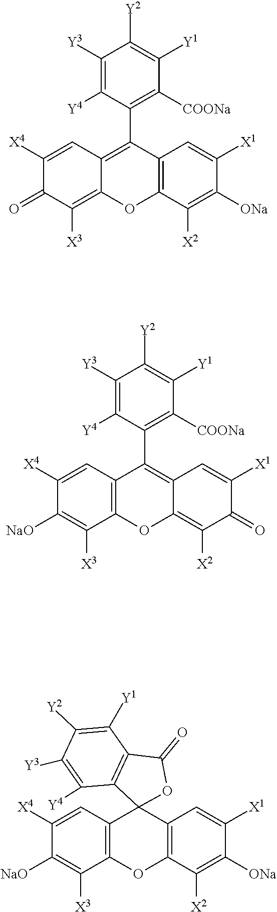

Examples of xanthene dyes according to a preferable embodiment of the present disclosure or salts thereof include rose bengal (Acid Red 94), eosin Y (tetrabromofluorescein, Solvent Red 43), Acid Red 87 (eosin Y disodium salt), erythrosin B (Acid Red 51), phloxine B (Acid Red 92), tetrabromofluorescein potassium, 2′,4′,5′,7′-tetrabromo-3,4,5,6-tetrachlorofluorescein (Solvent Red 48), tetraiodofluorescein (iodeosin), eosin B (Acid Red 91), dibromofluorescein (Solvent Red 72), and diiodofluorescein (Solvent Red 73). Examples of xanthene dyes according to a particular embodiment of the present disclosure include rose bengal (Acid Red 94), eosin Y (tetrabromofluorescein, Solvent Red 43), Acid Red 87 (eosin Y disodium salt), erythrosin B (Acid Red 51), phloxine B (Acid Red 92), tetrabromofluorescein potassium, 2′,4′,5′,7′-tetrabromo-3,4,5,6-tetrachlorofluorescein (Solvent Red 48), and tetraiodofluorescein (iodeosin). The xanthene dye according to the particular embodiment of the present disclosure may be rose bengal, eosin Y, or erythrosin B.

In one embodiment of the present disclosure, the concentration of the xanthene dye contained in the solution may be 0.01 to 100 μM, 0.1 to 50 μM, 0.2 to 25 μM, 0.3 to 10 μM, 0.4 to 6.0 μM, or 0.5 to 2.0 μM, for example.

The solution according to the first aspect of the present disclosure, for which the light-absorption/fluorescence characteristic(s) as an index of evaluation is measured, may further contain other components. Examples of the other components include pH adjusters, buffering agents, antioxidants, and aggregation promoters. The above-mentioned solution according to one embodiment of the present disclosure may contain only one type of the other components or may contain two or more types thereof.

A pH adjuster can adjust the pH of the solution to a range suitable for maintaining the aggregation state of the aggregative proteins and detecting the fluorescence of the xanthene dye by acidifying or basicizing the pH of the solution. In addition, the pH of the solution can be adjusted to a pH that promotes aggregation of the aggregative proteins. Examples of pH adjusters include acids such as hydrochloric acid, sulfuric acid, acetic acid, citric acid, lactic acid, phosphoric acid, tartaric acid, methanesulfonic acid, maleic acid, fumaric acid, and ammonium chloride; and bases such as lithium hydroxide, sodium hydroxide, magnesium hydroxide, aluminum hydroxide, potassium hydroxide, calcium hydroxide, sodium carbonate, sodium hydrogen carbonate, potassium carbonate, potassium hydrogen carbonate, calcium carbonate, cesium carbonate, ammonia, and methylamine.

A buffering agent can maintain the pH of the solution to a range suitable for maintaining the aggregation state of the aggregative proteins and detecting the fluorescence of the xanthene dye by a buffering action. Examples of buffering agents include phosphate buffers, Tris buffers, HEPES buffers, carbonate buffers, citrate buffers, borate buffers, and acetate buffers.

An antioxidant inhibits oxidation of the proteins, the xanthene dye, and the like in the solution by acting as a so-called active oxygen scavenger that inactivates singlet oxygen (¹O₂) produced by the xanthene dye upon irradiation with excitation light. Examples of antioxidants include ascorbic acid, α-tocopherol, catechin, glutathione, sodium azide (NaN₃), and 1,4-diazabicyclo[2.2.2]octane (DABCO).

An aggregation promoter is a component that promotes protein aggregation. Examples of aggregation promoters include substances that promote protein denaturation, such as guanidine, urea, and compounds having a guanidino group or an ureido group as a partial structure.

In one embodiment of the present disclosure, the total content of the other components may be 0.001% to 50% by mass, 0.01% to 20% by mass, 0.05% to 5.0% by mass, or 0.1% to 2.0% by mass, for example, based on the mass of the entire solution mentioned above.

The evaluation method according to the first aspect of the present disclosure includes evaluating the aggregation state of the aggregative proteins using, as an index, the light-absorption/fluorescence characteristic(s) of the xanthene dye in the solution containing the aggregative proteins and the xanthene dye or a salt thereof. In the solution containing the aggregative proteins and containing the xanthene dye or a salt thereof, the light-absorption/fluorescence characteristic(s) of the xanthene dye changes depending on the aggregation state of the aggregative proteins.

Therefore, the aggregation state of the aggregative proteins can be evaluated using the light-absorption/fluorescence characteristic(s) of the xanthene dye as an index.

In one embodiment of the present disclosure, the light-absorption/fluorescence characteristic(s) may include at least one selected from the group consisting of a fluorescence spectrum, a fluorescence intensity, a maximum fluorescence wavelength, a fluorescence lifetime, a fluorescence quantum yield, an absorption spectrum, an absorbance, and a maximum absorption wavelength, for example. In a preferable embodiment, the light-absorption/fluorescence characteristic(s) may include at least one selected from the group consisting of a fluorescence spectrum, a fluorescence intensity, a maximum fluorescence wavelength, an absorption spectrum, an absorbance, and a maximum absorption wavelength. In a more preferable embodiment, the light-absorption/fluorescence characteristic(s) may include at least one selected from the group consisting of a fluorescence spectrum, a fluorescence intensity, and a maximum fluorescence wavelength. In a particular embodiment, the light-absorption/fluorescence characteristic(s) may include at least one selected from the group consisting of a fluorescence spectrum, a maximum fluorescence intensity, and a maximum absorbance. In another particular embodiment, the light-absorption/fluorescence characteristic(s) may include at least one selected from the group consisting of a fluorescence spectrum and a fluorescence intensity, or may be a fluorescence spectrum or a fluorescence intensity.

In one embodiment of the present disclosure, the aggregation state of the aggregative proteins in the solution is evaluated based on a relationship between a time from when the aggregative proteins are brought into an aggregatable state in the solution, and the light-absorption/fluorescence characteristic(s) of the xanthene dye. For example, when the aggregative proteins are proteins that aggregate with each other under physiological conditions, the time from when brought into an aggregatable state means a time from when the aggregative proteins are added to an aqueous solvent under physiological conditions. In addition, for example, when the aggregative proteins are proteins that aggregate when heat denaturation occurs, the time from when brought into an aggregatable state means a time during which a high temperature state is maintained from the start of heating the solution containing the aggregative proteins. Furthermore, for example, when the aggregative proteins are proteins that start aggregating when a stimulus such as strong stirring and ultrasonic irradiation is applied thereto, the time from when brought into an aggregatable state means a time from when a stimulus is applied to the solution containing the aggregative proteins.

Regarding a method for evaluating the aggregation state, for example, when the absorption spectrum of the xanthene dye shifts in a direction in which a maximum absorption wavelength becomes longer and a maximum absorbance increases depending on the time from when the aggregative proteins are brought into an aggregatable state in the solution, the aggregation state of the aggregative proteins in the above-mentioned solution can be evaluated based on a relationship in which the absorption spectrum shifts in a time-dependent manner and a relationship in which the maximum absorption wavelength becomes longer. In addition, the aggregation state of the aggregative proteins in the above-mentioned solution can be evaluated based on a relationship in which a maximum absorbance, or an absorbance at a predetermined wavelength (for example, the arithmetic average wavelength of the maximum absorption wavelength before aggregation and after aggregation of proteins), which can be determined from an absorption spectrum, increases monotonically in a time-dependent manner.

For example, when the absorption spectrum of the xanthene dye shifts in a direction in which a maximum absorption wavelength becomes longer and in a direction in which a maximum absorbance decreases once and thereafter increases depending on the time from when the aggregative proteins are brought into an aggregatable state in the solution, the aggregation state of the aggregative proteins in the above-mentioned solution can be evaluated based on a relationship in which the absorption spectrum shifts in a time-dependent manner and a relationship in which the maximum absorption wavelength becomes longer. In addition, the aggregation state of the aggregative proteins in the above-mentioned solution can be evaluated based on a relationship in which a maximum absorbance, or an absorbance at a predetermined wavelength (for example, the arithmetic average wavelength of the maximum absorption wavelength before aggregation and after aggregation of proteins), which can be determined from an absorption spectrum, decreases and thereafter increases in a time-dependent manner.

For example, when the fluorescence spectrum of the xanthene dye shifts in a direction in which a maximum fluorescence wavelength becomes longer and in a direction in which a maximum fluorescence intensity decreases once and thereafter increases depending on the time from when the aggregative proteins are brought into an aggregatable state in the solution, the aggregation state of the aggregative proteins in the above-mentioned solution can be evaluated based on a relationship in which the fluorescence spectrum shifts in a time-dependent manner and a relationship in which the maximum fluorescence wavelength becomes longer. In addition, the aggregation state of the aggregative proteins in the above-mentioned solution can be evaluated based on a relationship in which a maximum fluorescence intensity, or a fluorescence intensity at a predetermined wavelength (for example, the arithmetic average wavelength of the maximum fluorescence wavelength before aggregation and after aggregation of proteins), which can be determined from a fluorescence spectrum, decreases and thereafter increases in a time-dependent manner.

In these cases, the above-mentioned predetermined wavelength can be determined depending on the xanthene dye and the aggregative proteins, and based on time-dependent changes in the shape of an absorption spectrum or a fluorescence spectrum. For example, the above-mentioned predetermined wavelength may be determined as a wavelength at which an absorbance or a fluorescence increases monotonically in a time-dependent manner. For example, the above-mentioned predetermined wavelength may be determined as a wavelength at which an absorbance or a fluorescence decreases and thereafter increases in a time-dependent manner, for example. For example, a wavelength at which an absorbance or a fluorescence decreases and thereafter increases in a time-dependent manner may be a wavelength near the arithmetic average wavelength of the maximum absorption wavelength or the maximum fluorescence wavelength before aggregation and after aggregation of proteins (for example, a wavelength within a range of ±5 nm from the arithmetic average wavelength).

In these cases, when the light-absorption/fluorescence characteristic(s) monotonically increases or monotonically decreases in a time-dependent manner, the aggregation state may be evaluated using, as an index, an inflection point of the light-absorption/fluorescence characteristic(s) with respect to time. Furthermore, when the light-absorption/fluorescence characteristic(s) decreases and thereafter increases according to time, the aggregation state may be evaluated using, as an index, a minimum point of the light-absorption/fluorescence characteristic(s) with respect to time, or may be evaluated using, as indexes, a minimum point and an inflection point of the light-absorption/fluorescence characteristic(s) with respect to time, for example. Through such evaluation, the aggregation state can be evaluated as two or more, three or more, or four or more stages of aggregation from a monomer state before forming aggregates to a final formation of fibrils via an intermediate state, for example.

In one embodiment of the present disclosure, the aggregation state of the aggregative proteins in the solution may be evaluated using, as a reference, known information about the light-absorption/fluorescence characteristic(s) of the xanthene dye depending on the aggregation state of proteins in the solution. For example, the light-absorption/fluorescence characteristic(s) may be an absorption spectrum or a fluorescence spectrum. In this case, the aggregation state of the aggregative proteins in the solution may be evaluated based on the condition in which the shape of the spectrum in a solution to be evaluated matches the shape of the spectrum in a specific aggregation state of the reference, for example. Furthermore, the light-absorption/fluorescence characteristic(s) in this case may be a maximum absorption wavelength or a maximum fluorescence wavelength, for example. In this case, the aggregation state of the aggregative proteins in the solution may be evaluated based on the condition in which the maximum absorption wavelength or the maximum fluorescence wavelength in a solution to be evaluated matches the maximum absorption wavelength or the maximum fluorescence wavelength in a specific aggregation state of the reference, for example. Furthermore, the light-absorption/fluorescence characteristic(s) in this case may be an absorbance or a fluorescence intensity at two or more wavelengths, for example. In this case, the aggregation state of the aggregative proteins in the solution may be evaluated based on the condition in which the ratio of the absorbance or the fluorescence intensity of the first wavelength and the second wavelength in a solution to be evaluated matches the ratio of the absorbance or the fluorescence intensity of the first wavelength and the second wavelength in a specific aggregation state of the reference, for example. Through such evaluation, the aggregation state can be evaluated as two or more, three or more, or four or more stages of aggregation from a monomer state before forming aggregates to a final formation of fibrils via an intermediate state, for example.

In one embodiment, the method according to the first aspect of the present disclosure includes: a step (I) of preparing an aqueous solution of aggregative proteins (preparation step); a step (II) of incubating the protein solution obtained in the preparation step to cause the aggregative proteins to aggregate (aggregation step); a step (III) of adding a xanthene dye or a salt thereof to a solution containing protein aggregates obtained in the aggregation step (dye addition step); a step (IV) of measuring the light-absorption/fluorescence characteristic(s) of the solution obtained in the dye addition step (measurement step); and a step (V) of evaluating the aggregation state of the aggregative proteins in the solution based on the light-absorption/fluorescence characteristic(s) acquired in the measurement step (evaluation step).

In the preparation step (I), an aqueous solution of aggregative proteins is prepared. The preparation step (I) is usually performed by adding the aggregative proteins to an aqueous solvent. In one embodiment, when the aggregative proteins are proteins that aggregate with each other under physiological conditions, aggregation starts when the aggregative proteins are dissolved in an aqueous solvent. In this case, it is preferable that the preparation step (I) be carried out immediately before the aggregation step (II) to be described later, or be carried out under ice-cooling as a condition under which aggregation does not occur, for example. Furthermore, the aqueous solvent may contain the above-mentioned other components.

In the aggregation step (II), the protein solution obtained in the preparation step (I) is incubated to cause the aggregative proteins to aggregate. In one embodiment, when the aggregative proteins are proteins that aggregate with each other under physiological conditions, the incubation condition in the aggregation step (II) may be a physiological condition, that is, a condition in which stirring is gently performed at room temperature or in an environment of equal to or higher than 20° C. and lower than 40° C., for example. Furthermore, in one embodiment, when the aggregative proteins are proteins that aggregate with each other by heat denaturation, the incubation condition in the aggregation step (II) may be a condition in which stirring is gently performed in an environment of 40° C. or higher and 90° C. or lower, or 50° C. or higher and 80° C. or lower, for example.

In the dye addition step (III), the xanthene dye or a salt thereof is added to the solution containing the protein aggregates obtained in the aggregation step (II). As the addition of the xanthene dye or a salt thereof, for example, the addition may be performed as a powder of the xanthene dye or a salt thereof, or the addition may be performed as a solution in which the xanthene dye or a salt thereof has been dissolved in an aqueous solution or an organic solvent such as dimethyl sulfoxide and ethanol. Furthermore, the xanthene dye or a salt thereof in the dye addition step (III) may be added as it is to the solution containing the protein aggregates obtained in the aggregation step (II), or may be added to a solution in which the solution containing the protein aggregates obtained in the aggregation step (II) has been diluted. When the xanthene dye or a salt thereof is added to the solution in which the solution containing the protein aggregates obtained in the aggregation step (II) has been diluted, the concentration of the aggregative proteins in the diluted solution may be 0.1 to 100 μM, 0.5 to 60 μM, 1.0 to 30 μM, or 2.0 to 20 μM, for example, based on monomers. Furthermore, the above-mentioned aqueous solvent may be used as a diluent solution for dilution.

The measurement step (IV) is a step of measuring the light-absorption/fluorescence characteristic(s) of the above-mentioned xanthene dye in the solution containing the aggregative proteins obtained in the dye addition step (III) and containing the xanthene dye or a salt thereof. The measurement step (IV) may be performed only once, or may be performed multiple times at time intervals (for example, 2 to 1,000 times at intervals of 10 seconds to 20 minutes). When the measurement step (IV) is performed multiple times, this means the case in which the measurement step (IV) and the aggregation step (II) are repeated alternately. The interval at which the measurement step (IV) is performed means the incubation time of the aggregation step (II) performed at each time of performing the measurement step (IV). The light-absorption/fluorescence characteristic(s) can be measured by a method commonly performed by those skilled in the art, and can be measured using an absorption spectrometer, a spectrofluorometer, a microwell plate reader, a fluorescence lifetime measurement device, or the like, for example. In addition, an excitation wavelength in fluorescence intensity measurement and fluorescence lifetime measurement can be determined according to the type of xanthene dye by a method commonly performed by those skilled in the art. For example, an excitation wavelength may be determined such that a maximum fluorescence intensity, or a fluorescence intensity at a predetermined wavelength that can be determined from the fluorescence spectrum decreases and thereafter increases in a time-dependent manner.

In the evaluation step (V), the aggregation state of the aggregative proteins in the solution is evaluated based on the light-absorption/fluorescence characteristic(s) obtained in the measurement step (IV). As already described above, the aggregation state can be evaluated by a method of performing evaluation based on a relationship between a time from when the aggregative proteins are brought into an aggregatable state in the solution, and the light-absorption/fluorescence characteristic(s) of the xanthene dye; a method of performing evaluation using, as a reference, known information about the light-absorption/fluorescence characteristic(s) of the xanthene dye depending on the aggregation state of proteins in the solution; and the like, for example.

In the example explained above, the aggregation step (II) is performed after the preparation step (I), but when the aggregative proteins are proteins that aggregate under specific conditions other than heating, a step (VI) of applying a stimulus that starts aggregation to the solution containing the proteins obtained in the preparation step (aggregation start step) may be further included after the preparation step (I) and before the aggregation step (II). The stimulus in the aggregation start step can be determined depending on aggregative proteins, and may be strong stirring using a vortex mixer, ultrasonic irradiation, acidification or basicization of pH, addition of an aggregation promoter, or the like, for example. Furthermore, in one embodiment, the aggregation start step (VI) may be performed at the same time with the preparation step (I). In this case, by preliminarily acidifying or basicizing the pH of an aqueous solvent used in the preparation step (I), or by preliminarily adding an aggregation promoter to an aqueous solvent used in the preparation step (I), the aggregation start step (VI) is performed at the same time with the preparation step (I), thereby causing aggregation to start immediately after adding the aggregative proteins to the aqueous solvent.

In addition, in the example explained above, the dye addition step (III) is performed after the aggregation step (II) and before the measurement step (IV), but the dye addition step (III) may be performed at an arbitrary timing before the measurement step (IV), that is, before the preparation step (I), at the same time with the preparation step (I), after the preparation step (I) and before the aggregation step (II), or at the same time with the aggregation step (II), for example.

In addition, in the example explained above, time-dependent changes in the light-absorption/fluorescence characteristic(s) of the xanthene dye are acquired by a method of alternately performing the aggregation step (II) and the measurement step (IV), but solutions for use in the measurement step (IV) (measurement solutions) may be extracted multiple times from the solution after a lapse of a predetermined incubation time in the aggregation step (II) to acquire time-dependent changes of the light-absorption/fluorescence characteristic(s) of the xanthene dye from the relationship between the incubation time in each of the extracted measurement solutions, and the light-absorption/fluorescence characteristic(s) of these measurement solutions. In other words, the evaluation method in one embodiment may include multiple times of a step (VII) of extracting a part of the solution containing the aggregative proteins as a measurement solution in a predetermined incubation time of the aggregation step (measurement solution extraction step). When the evaluation method according to one embodiment includes the measurement solution extraction step (VII), steps performed after the measurement solution extraction step (VII), such as the dye addition step (III) and the measurement step (IV), are performed using the measurement solution instead of the solution in the aggregation step (II). For example, when the measurement solution extraction step (VII) is performed 11 times at 10-minute intervals in the aggregation step (II) performed for 100 minutes to perform the dye addition step (III) and the measurement step (IV) on the obtained 11 measurement solutions, it is possible to obtain the light-absorption/fluorescence characteristic(s) corresponding to incubation times of 0, 10, 20, 30, 40, 50, 60, 70, 80, 90, and 100 minutes in the aggregation step (II). When the evaluation method according to one embodiment includes the measurement solution extraction step (VII) to acquire time-dependent changes in the light-absorption/fluorescence characteristic(s) of the xanthene dye, it is preferable to perform the dye addition step (III) and the measurement step (IV) immediately after the measurement solution extraction step (VII) to prevent further changes in the aggregation state in the measurement solutions from occurring, or it is preferable to store the measurement solutions under conditions in which further aggregation is not caused (for example, ice-cooling conditions, freezing conditions, acidic conditions, or basic conditions).

Furthermore, in the example explained above, the solution containing protein aggregates is prepared in the preparation step (I) and the aggregation step (II), but a method for preparing the solution containing protein aggregates is not limited thereto. In other words, instead of the solution prepared in the preparation step (I) and the aggregation step (II), for example, a solution containing aggregates of proteins obtained from a target, or a solution on which the aggregation step (II) has been performed using a solution containing proteins obtained from a target may be used. Examples of such solutions containing protein aggregates or solutions containing proteins include plasma, serum, cerebrospinal fluid, and lymph fluid, which are collected from animals such as humans, mice, and rats, and diluent solutions thereof.

A second aspect of the present disclosure is a kit comprising the xanthene dye or a salt thereof in the method for evaluating the aggregation state according to the first aspect of the present disclosure. A kit according to one embodiment of the second aspect of the present disclosure may comprise: the xanthene dye or a salt thereof in the method for evaluating the aggregation state according to the first aspect of the present disclosure; and an instruction manual describing evaluation of the aggregation state of the aggregative proteins in the solution using the light-absorption/fluorescence characteristic(s) of the xanthene dye as a criterion. A kit according to another embodiment of the second aspect of the present disclosure may comprise: the xanthene dye or a salt thereof in the method for evaluating the aggregation state according to the first aspect of the present disclosure; and at least one selected from the group consisting of an antioxidant substance, a pH adjuster, and a buffering agent.

For example, the kit according to the second aspect of the present disclosure can be used as a research reagent for elucidating functions and properties of aggregative proteins in each aggregation state or elucidating the evaluation and the like of aggregating properties of aggregative proteins depending on solution conditions by the method according to the first aspect of the present disclosure.

A third aspect of the present disclosure is a method for screening drugs that induce a change in the aggregation state of aggregative proteins or induce decomposition of an aggregate of the aggregative proteins, the method comprising evaluating the aggregation state of the aggregative proteins in a solution according to the method for evaluating the aggregation state according to the first aspect of the present disclosure.

A fourth aspect of the present disclosure is a method for evaluating drugs that induce a change in the aggregation state of aggregative proteins or induce decomposition of an aggregate of the aggregative proteins, the method comprising evaluating the aggregation state of the aggregative proteins in a solution according to the method for evaluating the aggregation state according to the first aspect of the present disclosure.

The method for screening or evaluating drugs that induce a change in the aggregation state of the aggregative proteins according to the third and fourth aspects of the present disclosure can be performed by a method for further adding a drug to be screened or a drug to be evaluated to the aqueous solvent used in the preparation step (I) in the first aspect of the present disclosure, for example.

The method for screening or evaluating drugs that induce decomposition of the aggregation state of the aggregative proteins according to the third and fourth aspects of the present disclosure can be performed by adding a drug to be screened or a drug to be evaluated to the solution obtained in the aggregation step (II) in the first aspect of the present disclosure, and performing the measurement step (IV) after incubating for a predetermined time, for example.

Examples of the drugs according to the third and fourth aspects of the present disclosure include therapeutic agents for diseases induced by protein aggregates, such as Alzheimer's disease, Parkinson's disease, prion diseases, amyotrophic lateral sclerosis, Huntington's disease, and amyloidosis, and candidate substances thereof.

A fifth aspect of the present disclosure is a screening kit for use in the screening method according to the third aspect of the present disclosure, the screening kit comprising: the xanthene dye or a salt thereof in the method for evaluating the aggregation state according to the first aspect of the present disclosure; and aggregative proteins.

A sixth aspect of the present disclosure is an evaluation kit for use in the method for evaluating drugs according to the fourth aspect of the present disclosure, the evaluation kit comprising: the xanthene dye or a salt thereof in the method for evaluating the aggregation state according to the first aspect of the present disclosure; and aggregative proteins.

In the fifth and sixth aspects of the present disclosure, examples of combinations of drugs and aggregative proteins include a combination of a therapeutic agent for Alzheimer's disease or a candidate substance thereof, and lysozyme; a combination of a therapeutic agent for Alzheimer's disease or a candidate substance thereof, and amyloid β; a combination of a therapeutic agent for Parkinson's disease or a candidate substance thereof, and α-synuclein; a combination of a therapeutic agent for prion diseases or a candidate substance thereof, and prion protein; a combination of a therapeutic agent for amyotrophic lateral sclerosis or a candidate substance thereof, and Superoxide Dismutase 1 (SOD1); a combination of a therapeutic agent for amyotrophic lateral sclerosis or a candidate substance thereof, and TAR DNA-binding protein of 43 kDa (TDP-43); and a combination of a therapeutic agent for Huntington's disease or a candidate substance thereof, and polyglutamine.

In one embodiment of the present disclosure, the methods according to the third and fourth aspects of the present disclosure and the kits according to the fifth and sixth aspects of the present disclosure can be used for screening or evaluating therapeutic agents for diseases induced by protein aggregates, such as Alzheimer's disease, Parkinson's disease, prion diseases, amyotrophic lateral sclerosis, Huntington's disease, and amyloidosis, for example.

A seventh aspect of the present disclosure is a method for isolating an aggregate of aggregative proteins, the method including isolating an aggregate of aggregative proteins from a solution using, as an index, the aggregation state of the aggregative proteins in the solution evaluated according to the method for evaluating the aggregation state according to the first aspect of the present disclosure. For example, the isolation of protein aggregates in the isolation method according to the seventh aspect of the present disclosure can be performed by providing a step of isolating after the evaluation step (V) from the solution used in the measurement step (IV), the solution obtained in the aggregation step (II), or the solution obtained in the measurement solution extraction step (VII) based on the evaluation results by a ultracentrifugation technique of protein aggregates, a centrifugation technique using a filter such as Amicon (registered trademark), a column chromatography technique, a freeze-drying technique, or the like (isolation step). For example, in the evaluation step (V) performed using the measurement solution obtained in the measurement solution extraction step (VII) performed at a certain incubation time in the aggregation step (II), when the aggregation state of the aggregative proteins in the measurement solution is evaluated to be a desired state, the aggregation step (II) is terminated at the incubation time to perform the isolation step, which makes it possible to obtain aggregates having a desired aggregation state. For example, such an incubation time may be determined in advance using another experiment carried out under similar conditions as a reference, or may be determined in real time by repeatedly performing the measurement solution extraction step (VII), the dye addition step (III), the measurement step (IV), and the evaluation step (V), in parallel with the aggregation step (II).

### EXAMPLES

The present disclosure will be described in more detail below using examples, but the present disclosure is not limited to the following examples.

### Example 1: Evaluation of Aggregation State of Lysozyme Using Light-Absorption/Fluorescence Characteristics of Rose Bengal as Index

40 mg of lysozyme (manufactured by FUJIFILM Wako Pure Chemical Corporation) was added to 2 mL of an aqueous solution of 25 mM of hydrochloric acid and 100 mM of sodium chloride, and dissolved by being left to stand. The obtained lysozyme solution was heat-stirred at 200 rpm at 60° C. to cause heat denaturation of the lysozyme, thereby starting the aggregation reaction of the lysozyme. At 0, 10, 20, 25, 30, 35, 40, 45, 50, 60, 70, 80, and 90 minutes after starting heat-stirring, each of 100 μL of the lysozyme solution was extracted and ice-cooled. The ice-cooled lysozyme solution, and rose bengal (manufactured by FUJIFILM Wako Pure Chemical Corporation) were added to distilled water such that final concentrations were each 7 μM and 1 μM. The obtained solution was put in a spectroscopic measurement cell to measure the light-absorption/fluorescence characteristics. The excitation wavelength in fluorescence measurement was 530 nm.

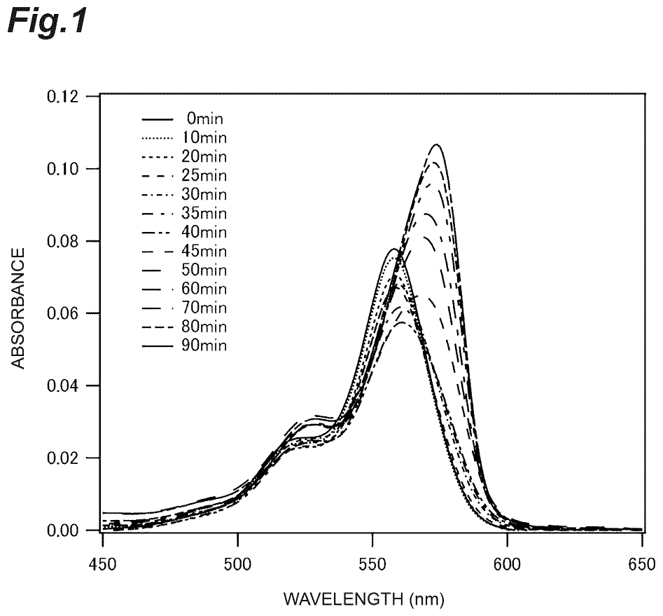

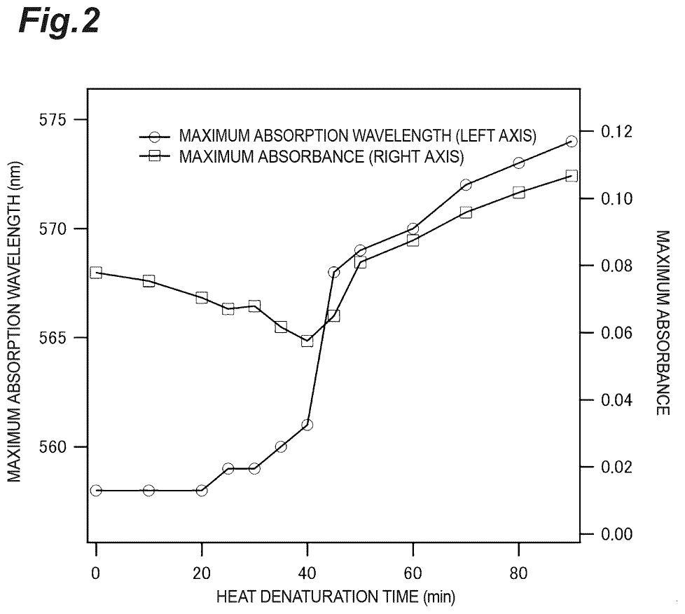

FIG. 1 shows absorption spectra at various heating-stirring times (heat denaturation times). Furthermore, FIG. 2 shows the results of plotting with a horizontal axis representing a heat denaturation time and a vertical axis representing a maximum absorption wavelength or a maximum absorbance in FIG. 1. According to FIGS. 1 and 2, the shape of the absorption spectra, the maximum absorption wavelength, and the maximum absorbance of rose bengal changed depending on a heat denaturation time. In particular, it was observed that the absorbance at around 560 nm as a predetermined wavelength that can be determined from the absorption spectra, and the maximum absorbance were changed from decreasing and thereafter increasing at around 40 to 50 minutes as a minimum point, with respect to the heat denaturation time. From the above results, it was shown that changes in the aggregation state of lysozyme depending on the heat denaturation time can be evaluated using the light absorption characteristic of rose bengal as an index.

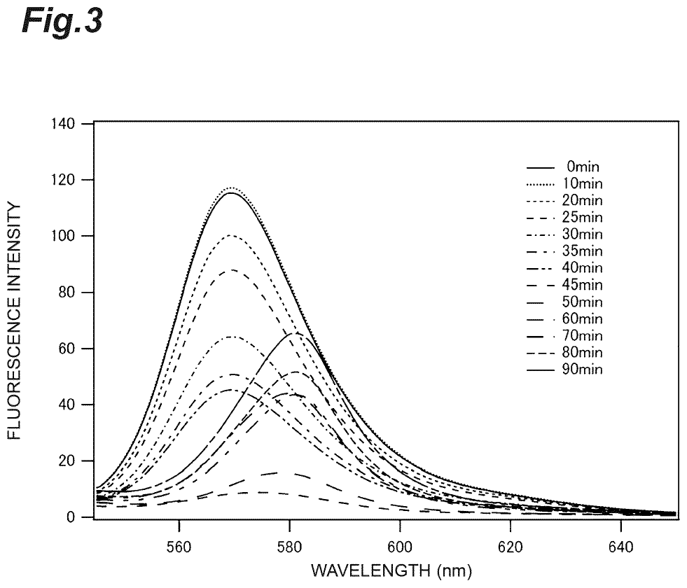

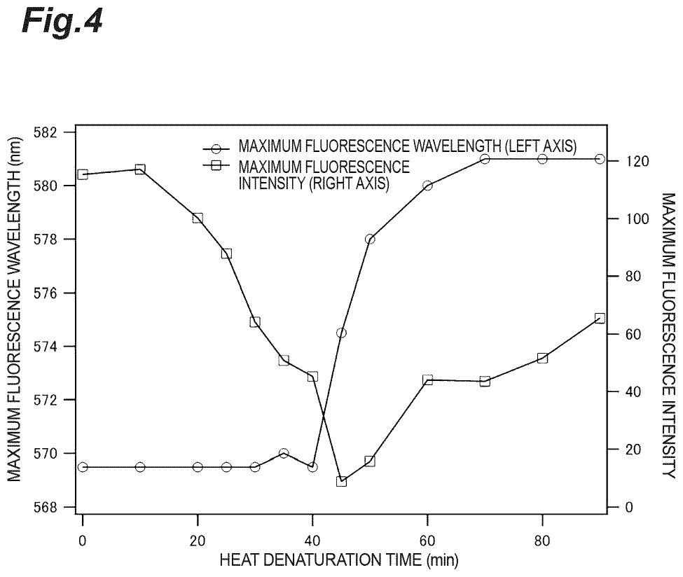

FIG. 3 shows fluorescence spectra at various heating-stirring times (heat denaturation times). Furthermore, FIG. 4 shows the results of plotting with a horizontal axis representing a heat denaturation time and a vertical axis representing a maximum fluorescence wavelength or a maximum fluorescence intensity in FIG. 3. According to FIGS. 3 and 4, the shape of the fluorescence spectra, the maximum fluorescence wavelength, and the maximum fluorescence intensity of rose bengal changed depending on a heat denaturation time. In particular, it was observed that the fluorescence intensity at around 570 nm as a predetermined fluorescence wavelength that can be determined from the fluorescence spectra, and the maximum fluorescence intensity were changed from decreasing and thereafter increasing at around 40 to 50 minutes as a minimum point, with respect to the heat denaturation time. From the above results, it was shown that changes in the aggregation state of lysozyme depending on the heat denaturation time can be evaluated using the fluorescence characteristic of rose bengal as an index.

### Example 2: Evaluation of Aggregation State of Insulin Using Light-Absorption/Fluorescence Characteristics of Rose Bengal as Index

40 mg of insulin (manufactured by FUJIFILM Wako Pure Chemical Corporation) was added to 2 mL of an aqueous solution of 25 mM of hydrochloric acid and 100 mM of sodium chloride, and dissolved by being left to stand. The obtained insulin solution was heat-stirred at 200 rpm at 55° C. to cause heat denaturation of the insulin, thereby starting the aggregation reaction of the insulin. At 0, 5, 10, 15, 20, 25, 30, 35, 40, 50, 60, 70, 80, and 90 minutes after starting heat-stirring, each of 100 μL of the insulin solution was extracted and ice-cooled. The ice-cooled insulin solution, and rose bengal (manufactured by FUJIFILM Wako Pure Chemical Corporation) were added to distilled water such that final concentrations were each 17 μM and 1 μM. The obtained solution was put in a spectroscopic measurement cell to measure the light-absorption/fluorescence characteristics. The excitation wavelength in fluorescence measurement was 530 nm.

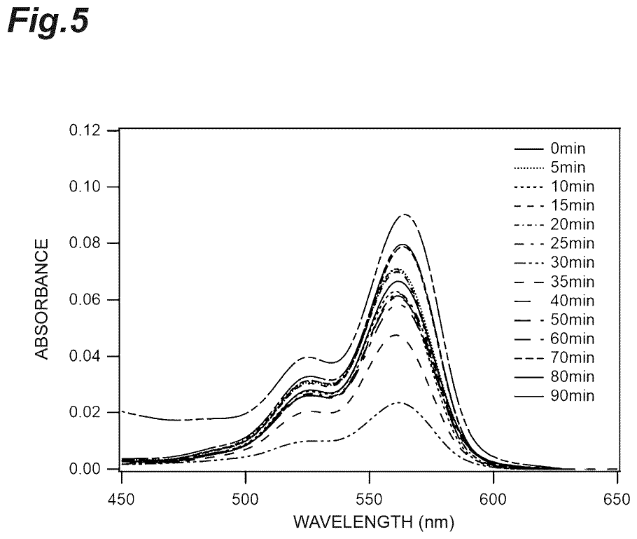

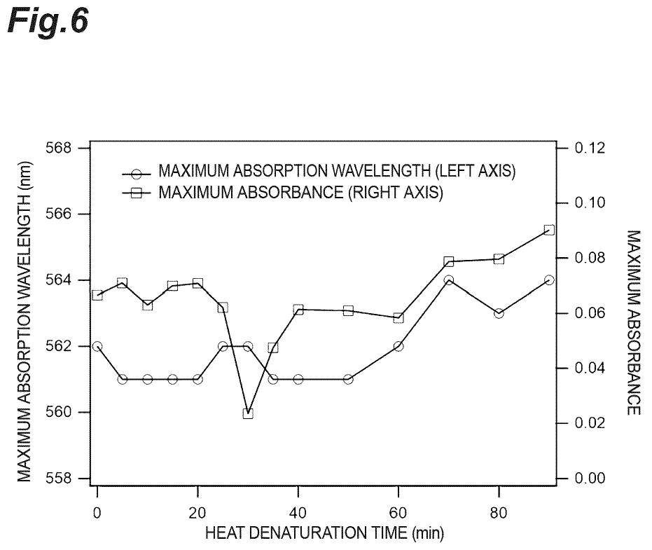

FIG. 5 shows absorption spectra at various heating-stirring times (heat denaturation times). Furthermore, FIG. 6 shows the results of plotting with a horizontal axis representing a heat denaturation time and a vertical axis representing a maximum absorption wavelength or a maximum absorbance in FIG. 5. According to FIGS. 5 and 6, the shape of the absorption spectra, the maximum absorption wavelength, and the maximum absorbance of rose bengal changed depending on a heat denaturation time. In particular, it was observed that the absorbance at around 560 nm as a predetermined wavelength that can be determined from the absorption spectra, and the maximum absorbance were changed from decreasing and thereafter increasing at around 30 minutes as a minimum point, with respect to the heat denaturation time. From the above results, it was shown that changes in the aggregation state of insulin depending on the heat denaturation time can be evaluated using the light absorption characteristic of rose bengal as an index.

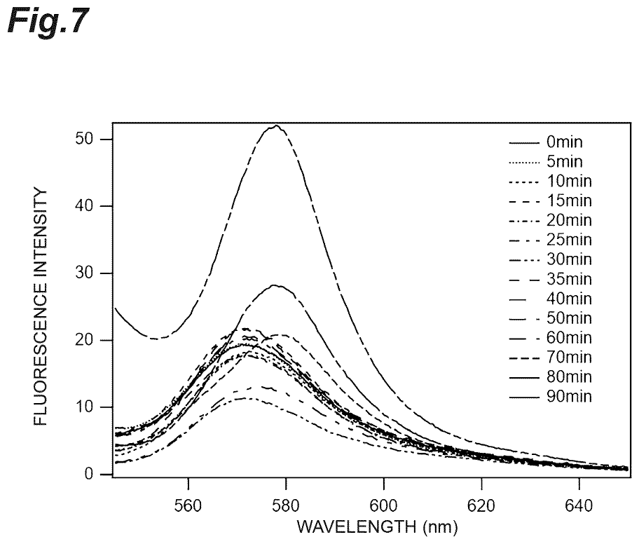

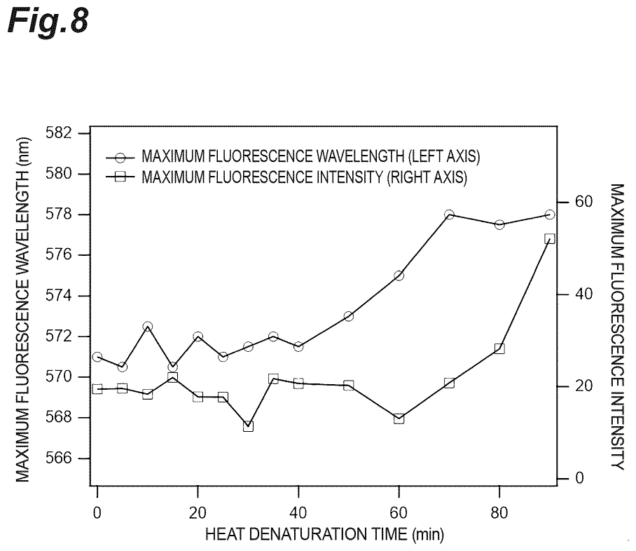

FIG. 7 shows fluorescence spectra at various heating-stirring times (heat denaturation times). Furthermore, FIG. 8 shows the results of plotting with a horizontal axis representing a heat denaturation time and a vertical axis representing a maximum fluorescence wavelength or a maximum fluorescence intensity in FIG. 7. According to FIGS. 7 and 8, the shape of the fluorescence spectra, the maximum fluorescence wavelength, and the maximum fluorescence intensity of rose bengal changed depending on a heat denaturation time. In particular, it was observed that the fluorescence intensity at around 570 nm as a predetermined fluorescence wavelength that can be determined from the fluorescence spectra, and the maximum fluorescence intensity were changed from decreasing and thereafter increasing at around 30 minutes as a minimum point, with respect to the heat denaturation time. From the above results, it was shown that changes in the aggregation state of insulin depending on the heat denaturation time can be evaluated using the fluorescence characteristic of rose bengal as an index.

### Example 3: Evaluation of Aggregation State of Horseradish Peroxidase Using Light-Absorption/Fluorescence Characteristics of Rose Bengal as Index

40 mg of horseradish peroxidase (HRP, manufactured by FUJIFILM Wako Pure Chemical Corporation) was added to 2 mL of phosphate buffered saline at pH 7.4, and dissolved by being left to stand. The obtained HRP solution was heat-stirred at 200 rpm at 70° C. to cause heat denaturation of the HRP, thereby starting the aggregation reaction of the HRP. At 0, 5, 10, 15, 20, 25, 30, 35, 40, 50, 60, 70, 80, and 90 minutes after starting heat-stirring, each of 100 μL of the HRP solution was extracted and ice-cooled. The ice-cooled HRP solution, and rose bengal (manufactured by FUJIFILM Wako Pure Chemical Corporation) were added to distilled water such that final concentrations were each 5 μM and 1 μM. The obtained solution was put in a spectroscopic measurement cell to measure the light-absorption/fluorescence characteristics. The excitation wavelength in fluorescence measurement was 530 nm.

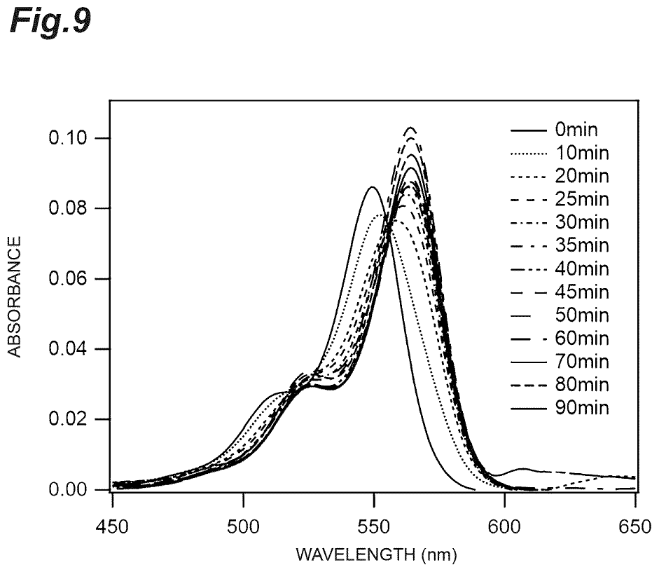

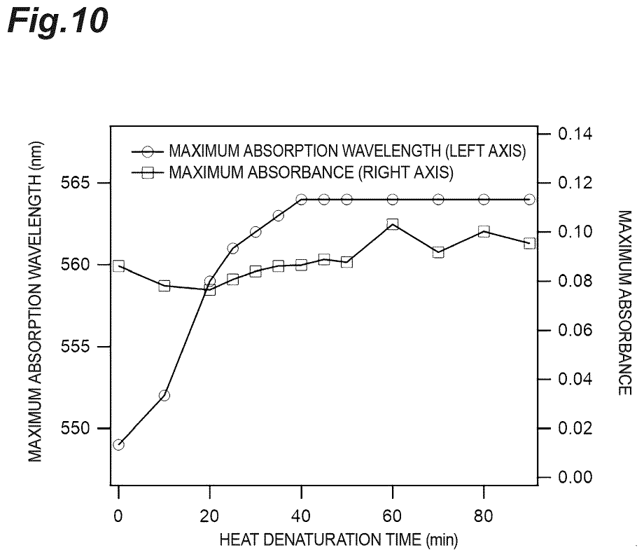

FIG. 9 shows absorption spectra at various heating-stirring times (heat denaturation times). Furthermore, FIG. 10 shows the results of plotting with a horizontal axis representing a heat denaturation time and a vertical axis representing a maximum absorption wavelength or a maximum absorbance in FIG. 9. In FIG. 9, because HRP has an absorption band that overlaps with the absorption of rose bengal, the absorption of HRP obtained as a reference to which rose bengal was not added was subtracted and displayed. According to FIGS. 9 and 10, the shape of the absorption spectra, the maximum absorption wavelength, and the maximum absorbance of rose bengal changed depending on a heat denaturation time. In particular, it was observed that the maximum absorbance was changed from decreasing and thereafter increasing at around 20 minutes as a minimum point, with respect to the heat denaturation time. From the above results, it was shown that changes in the aggregation state of HRP depending on the heat denaturation time can be evaluated using the light absorption characteristic of rose bengal as an index.

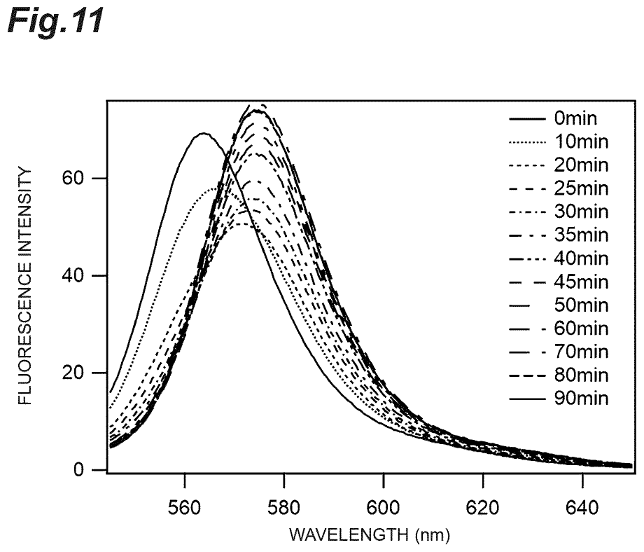

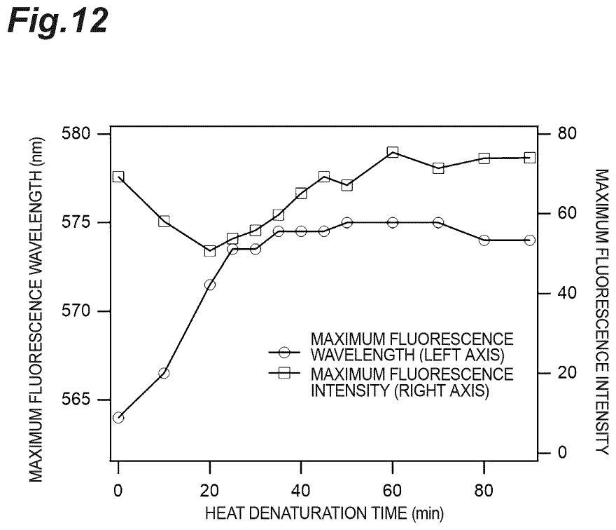

FIG. 11 shows fluorescence spectra at various heating-stirring times (heat denaturation times). Furthermore, FIG. 12 shows the results of plotting with a horizontal axis representing a heat denaturation time and a vertical axis representing a maximum fluorescence wavelength or a maximum fluorescence intensity in FIG. 11. According to FIGS. 11 and 12, the shape of the fluorescence spectra, the maximum fluorescence wavelength, and the maximum fluorescence intensity of rose bengal changed depending on a heat denaturation time. In particular, it was observed that the fluorescence intensity at around 570 nm as a predetermined fluorescence wavelength that can be determined from the fluorescence spectra, and the maximum fluorescence intensity were changed from decreasing and thereafter increasing at around 20 minutes as a minimum point, with respect to the heat denaturation time. From the above results, it was shown that changes in the aggregation state of HRP depending on the heat denaturation time can be evaluated using the fluorescence characteristic of rose bengal as an index.

### Example 4: Evaluation of Aggregation State of Lysozyme Using Light-Absorption/Fluorescence Characteristics of Eosin Y as Index

Sample preparation and light absorption and fluorescence measurement were performed in the same manner as in Example 1 except that eosin Y (manufactured by FUJIFILM Wako Pure Chemical Corporation) was used instead of rose bengal as the xanthene dye, and that the excitation wavelength in fluorescence measurement was set to 500 nm.

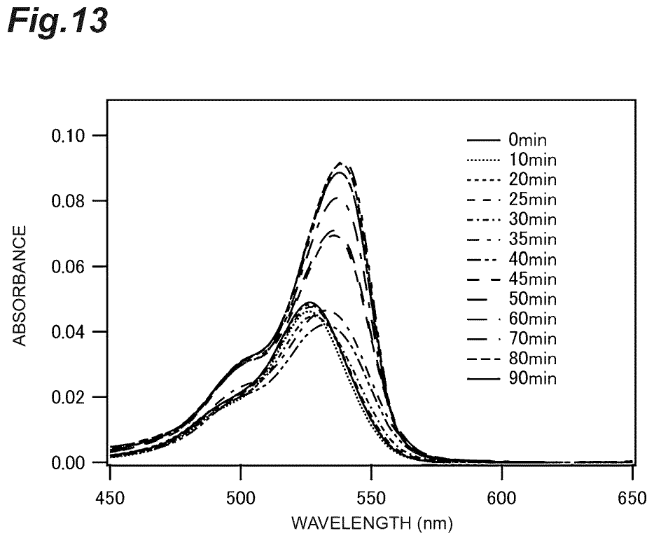

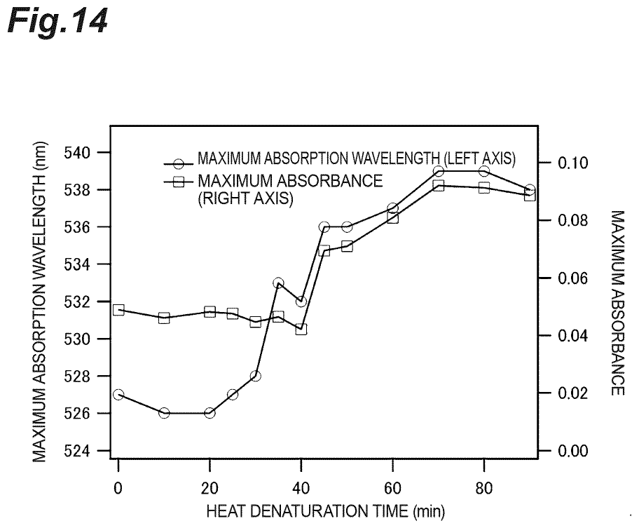

FIG. 13 shows absorption spectra at various heating-stirring times (heat denaturation times). Furthermore, FIG. 14 shows the results of plotting with a horizontal axis representing a heat denaturation time and a vertical axis representing a maximum absorption wavelength or a maximum absorbance in FIG. 13. According to FIGS. 13 and 14, the shape of the absorption spectra, the maximum absorption wavelength, and the maximum absorbance of eosin Y changed depending on a heat denaturation time. In particular, it was observed that the absorbance at around 530 nm as a predetermined wavelength that can be determined from the absorption spectra, and the maximum absorbance were changed from decreasing and thereafter increasing at around 40 to 50 minutes as a minimum point, with respect to the heat denaturation time. This time-dependent change was consistent with the results in FIG. 1 in which rose bengal was used. From the above results, it was shown that changes in the aggregation state of lysozyme depending on the heat denaturation time can be evaluated using the light absorption characteristic of eosin Y as an index.

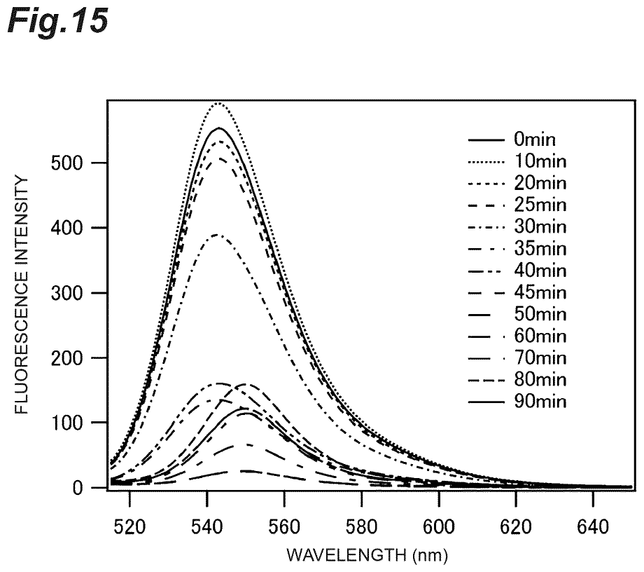

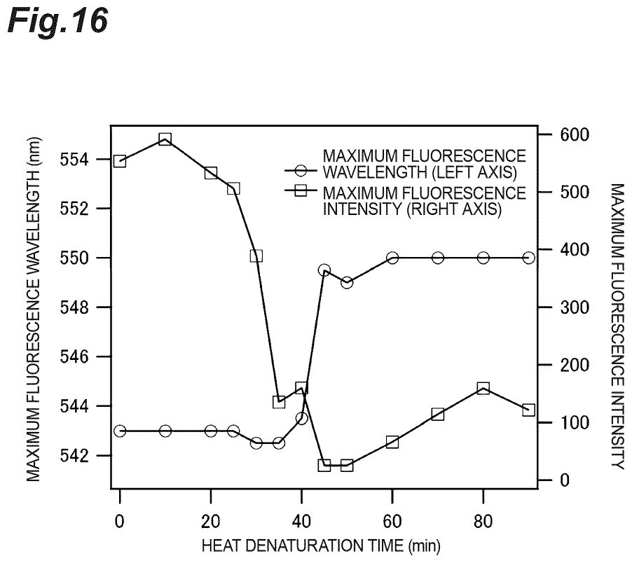

FIG. 15 shows fluorescence spectra at various heating-stirring times (heat denaturation times). Furthermore, FIG. 16 shows the results of plotting with a horizontal axis representing a heat denaturation time and a vertical axis representing a maximum fluorescence wavelength or a maximum fluorescence intensity in FIG. 15. According to FIGS. 15 and 16, the shape of the fluorescence spectra, the maximum fluorescence wavelength, and the maximum fluorescence intensity of eosin Y changed depending on a heat denaturation time. In particular, it was observed that the fluorescence intensity at around 550 nm as a predetermined fluorescence wavelength that can be determined from the fluorescence spectra, and the maximum fluorescence intensity were changed from decreasing and thereafter increasing at around 40 to 50 minutes as a minimum point, with respect to the heat denaturation time. This time-dependent change was consistent with the results in FIG. 1 in which rose bengal was used. From the above results, it was shown that changes in the aggregation state of lysozyme depending on the heat denaturation time can be evaluated using the fluorescence characteristic of eosin Y as an index.

### Example 5: Evaluation of Aggregation State of Lysozyme Using Light-Absorption/Fluorescence Characteristics of Erythrosin B as Index

Sample preparation and light absorption and fluorescence measurement were performed in the same manner as in Example 1 except that erythrosin B (manufactured by FUJIFILM Wako Pure Chemical Corporation) was used instead of rose bengal as the xanthene dye, and that the excitation wavelength in fluorescence measurement was set to 510 nm.

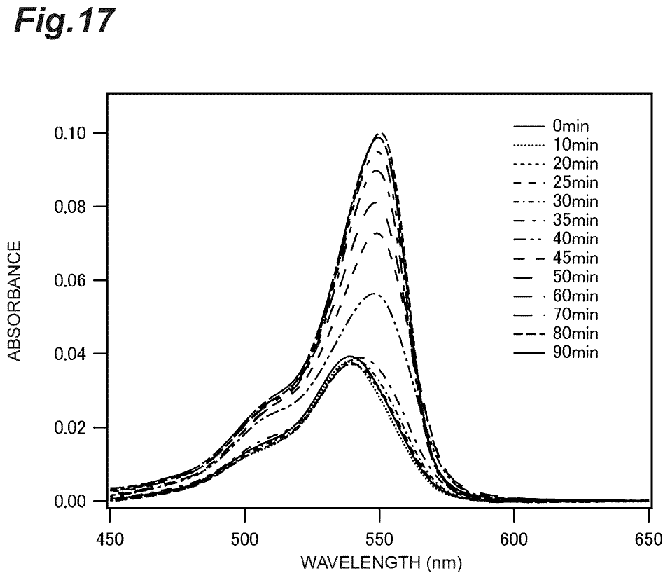

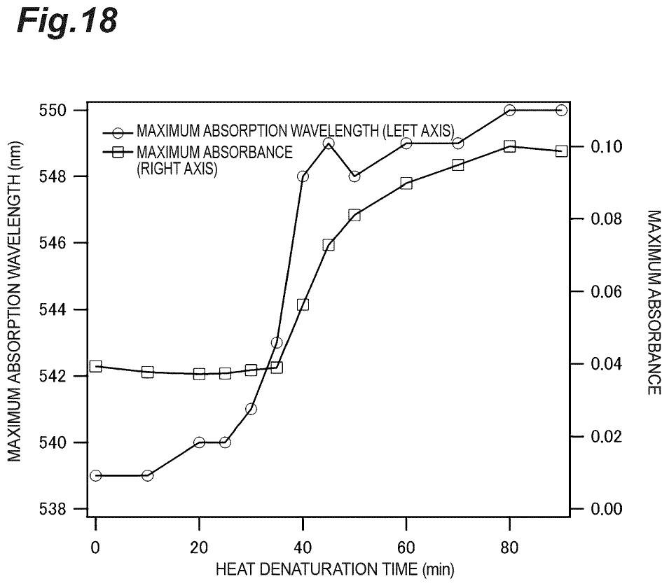

FIG. 17 shows absorption spectra at various heating-stirring times (heat denaturation times). Furthermore, FIG. 18 shows the results of plotting with a horizontal axis representing a heat denaturation time and a vertical axis representing a maximum absorption wavelength or a maximum absorbance in FIG. 17. According to FIGS. 17 and 18, the shape of the absorption spectra, the maximum absorption wavelength, and the maximum absorbance of erythrosin B changed depending on a heat denaturation time. From the above results, it was shown that changes in the aggregation state of lysozyme depending on the heat denaturation time can be evaluated using the light absorption characteristic of erythrosin B as an index.

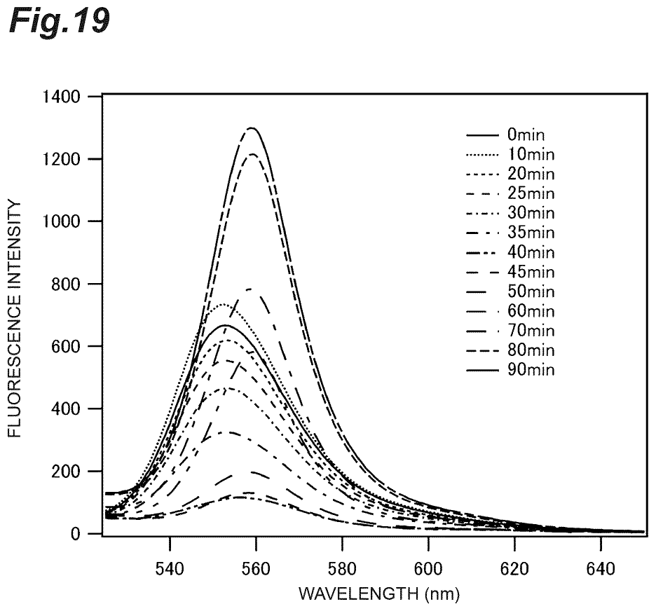

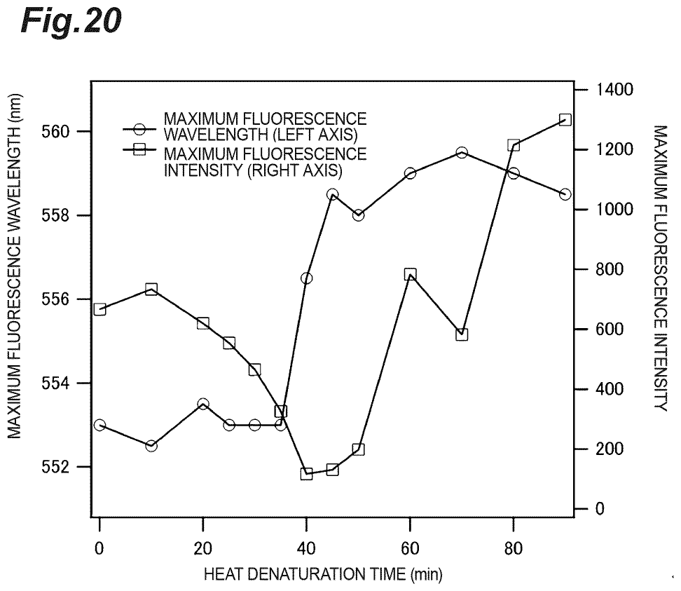

FIG. 19 shows fluorescence spectra at various heating-stirring times (heat denaturation times). Furthermore, FIG. 20 shows the results of plotting with a horizontal axis representing a heat denaturation time and a vertical axis representing a maximum fluorescence wavelength or a maximum fluorescence intensity in FIG. 19. According to FIGS. 19 and 20, the shape of the fluorescence spectra, the maximum fluorescence wavelength, and the maximum fluorescence intensity of erythrosin B changed depending on a heat denaturation time. In particular, it was observed that the fluorescence intensity at around 560 nm as a predetermined fluorescence wavelength that can be determined from the fluorescence spectra, and the maximum fluorescence intensity were changed from decreasing and thereafter increasing at around 40 to 50 minutes as a minimum point, with respect to the heat denaturation time. This time-dependent change was consistent with the results in FIG. 1 in which rose bengal was used. From the above results, it was shown that changes in the aggregation state of lysozyme depending on the heat denaturation time can be evaluated using the fluorescence characteristic of erythrosin B as an index.

What is claimed is:

1. A method for evaluating an aggregation state of aggregative proteins in a solution, the method comprising
evaluating an aggregation state of aggregative proteins using, as an index, light-absorption/fluorescence characteristic(s) of a xanthene dye in a solution comprising the aggregative proteins and the xanthene dye or a salt thereof,
wherein the xanthene dye is represented by Formula (I): 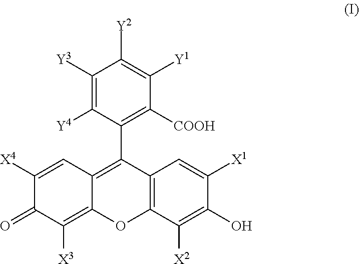
wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

2. The method according to claim 1, wherein the aggregation state is evaluated based on a relationship between a time from when the aggregative proteins are brought into an aggregatable state in the solution, and the light-absorption/fluorescence characteristic(s) of the xanthene dye.

3. The method according to claim 1, wherein X¹ to X⁴ are each independently —H, —OH, —NO₂, —F, —Cl, —Br, or —I, provided that at least two of X¹ to X⁴ are —Br or —I; and Y¹ to Y⁴ are each independently —H or —Cl.

4. The method according to claim 1, wherein X¹ to X⁴ are each independently —Br or —I; and Y¹ to Y⁴ are each independently —H or —Cl.

5. The method according to claim 1, wherein the light-absorption/fluorescence characteristic(s) includes at least one selected from the group consisting of a fluorescence spectrum, a fluorescence intensity, a maximum fluorescence wavelength, an absorption spectrum, an absorbance, and a maximum absorption wavelength.

6. The method according to claim 1, wherein the light-absorption/fluorescence characteristic(s) includes at least one selected from the group consisting of a fluorescence spectrum, a fluorescence intensity, and a maximum fluorescence wavelength.

7. The method according to claim 2, wherein the light-absorption/fluorescence characteristic(s) is a fluorescence spectrum or a fluorescence intensity,
and wherein the aggregation state of the aggregative proteins is evaluated based on a relationship in which a maximum fluorescence intensity or a fluorescence intensity at a predetermined fluorescence wavelength decreases and thereafter increases with respect to a time from when the aggregative proteins are brought into an aggregatable state in the solution, the predetermined fluorescence wavelength being a wavelength which can be determined depending on the xanthene dye and the aggregative proteins.

8. A kit for use in the method according to claim 1, the kit comprising:
the xanthene dye or the salt thereof; and
an instruction manual describing evaluation of an aggregation state of aggregative proteins in a solution using light-absorption/fluorescence characteristic(s) of the xanthene dye as a criterion,
wherein the xanthene dye is represented by Formula (I): 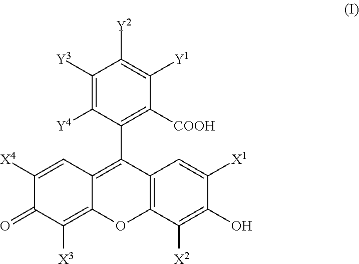
wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

9. A kit for use in the method according to claim 1, the kit comprising:
the xanthene dye or the salt thereof; and
at least one selected from the group consisting of an antioxidant substance, a pH adjuster, and a buffering agent,
wherein the xanthene dye is represented by Formula (I): 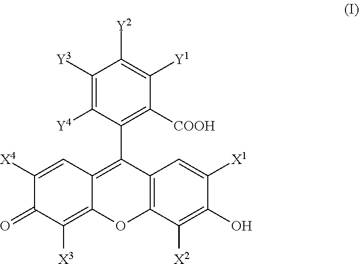
wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

10. A method for screening or evaluating drugs that induce a change in an aggregation state of aggregative proteins or induce decomposition of an aggregate of the aggregative proteins, the method comprising evaluating the aggregation state of the aggregative proteins in a solution according to the method according to claim 1.

11. A screening or evaluating kit for use in the method according to claim 10, the screening kit comprising:
the xanthene dye or the salt thereof; and
the aggregative protein,
wherein the xanthene dye is represented by Formula (I): 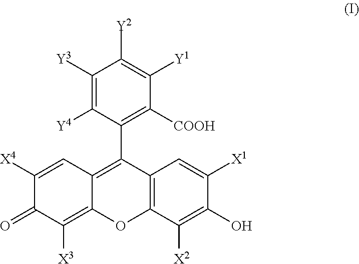
wherein X¹ to X⁴ are each independently —H, —OH, —NO₂, —F, —Cl, —Br, or —I, provided that at least one of X¹ to X⁴ is —Br or —I; and Y¹ to Y⁴ are each independently —H, —F, or —Cl.

12. A method for isolating an aggregate of aggregative proteins, the method comprising
isolating an aggregate of the aggregative proteins from the solution using, as an index, the aggregation state of the aggregative proteins in the solution evaluated according to the method according to claim 1.

13. The kit according to claim 8, wherein X¹ to X⁴ are each independently —H, —OH, —NO₂, —F, —Cl, —Br, or —I, provided that at least two of X¹ to X⁴ are —Br or —I; and Y¹ to Y⁴ are each independently —H or —Cl.

14. The kit according to claim 8, wherein X¹ to X⁴ are each independently —Br or —I; and Y¹ to Y⁴ are each independently —H or —Cl.

15. The kit according to claim 9, wherein X¹ to X⁴ are each independently —H, —OH, —NO₂, —F, —Cl, —Br, or —I, provided that at least two of X¹ to X⁴ are —Br or —I; and Y¹ to Y⁴ are each independently —H or —Cl.

16. The kit according to claim 9, wherein X¹ to X⁴ are each independently —Br or —I; and Y¹ to Y⁴ are each independently —H or —Cl.

17. The kit according to claim 11, wherein X¹ to X⁴ are each independently —H, —OH, —NO₂, —F, —Cl, —Br, or —I, provided that at least two of X¹ to X⁴ are —Br or —I; and Y¹ to Y⁴ are each independently —H or —Cl.

18. The kit according to claim 11, wherein X¹ to X⁴ are each independently —Br or —I; and Y¹ to Y⁴ are each independently —H or —Cl.

19. The kit according to claim 8, wherein the instruction manual describes evaluation of an aggregation state of aggregative proteins in accordance with the method according to the claim 2.

20. The kit according to claim 8, wherein the instruction manual describes evaluation of an aggregation state of aggregative proteins in accordance with the method according to the claim 7.
